(19) United States
(12) Patent Application Publication
(10) Pub. No.: US 2014/0309569 A1
(43) Pub. Date: Oct. 16, 2014

(54) Reusable Wound Care Dressing System

(71) Applicant: Diana Lynn Matthews, Tomball, TX (US)

(72) Inventor: Diana Lynn Matthews, Tomball, TX (US)

(21) Appl. No.: 13/862,141

(22) Filed: Apr. 12, 2013

(51) Int. Cl. A61F 13/00; A61F 5/37

(52) U.S. Cl. CPC A61F 13/00085; A61F 5/37; A61F 13/00063
USPC 602/4; 602/42; 602/48

(57) ABSTRACT

A dressing support system and method of use for bandaging patient wounds in an efficient, cost effective, and repeatable manner by practitioners, non-professionals, and home caregivers according to unique specification dictated by wound care specialist without the need for extensive training or wasted materials of trial and error techniques.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO SEQUENCE LISTING, A TABLE, OR A COMPUTER PROGRAM LISTING APPENDIX SUBMITTED ON COMPACT DISC

Not applicable.

### BACKGROUND OF THE INVENTION

The subject matter herein is directed to dressing and care of wounds. In particular, reusable dressings, which can be efficiently applied by non-professionals or dressings applied to areas of the body which are susceptible to difficulty in wound protection due to movement and use by the patient, i.e. difficult anatomical sites.

The correct dressing can have a significant impact on the healing of wounds. Experts have long understood the importance of maintaining the proper environment, based on the wound type, which promote the natural healing process. Matching wound characteristics with dressing features is one important goal in the healing process. For example, a heavily exudating wound needs an absorptive dressing, and a wound with necrotic eschar needs a dressing that facilitates debridement. Other factors may be controlling odor in fungating and malodorous wounds, and concealing wounds in prominent locations to avoid patient embarrassment or unwanted attention.

Practitioners can select and prepare a proper dressing, but that environment must be maintainable by the patient when out of the direct care of the Practitioner. Failure to properly dress wounds and maintain the healing environment can delay healing, and lead to chronic medical issues such as infection.

The primary purposes of dressing wounds are to control bleeding, removal and/or prevention of foreign bodies that could act as a focus for infection, removal of devitalized tissue, thick slough and pus, promote growth of granulation tissue and epithelialization, and to protect the wound from further trauma and pathogenic micro-organisms. Dressings do this through regulation of temperature, humidity and pH to optimum levels for the cells involved in the healing process.

The wide selection and sophistication of modern wound dressings available makes selection difficult. Multiple wounds in close proximity may make dressing even more complex. This is especially true when different wounds have sufficiently different pathological features as to require differing environments for optimal healing responses.

There are a number of principal reasons for dressing wounds. Proper dressing provides optimal environmental conditions for rapid and cosmetically acceptable healing. Dressings may contain or remove odor. Dressing protects sensitive areas and reduces pain. Proper dressings prevent contamination, which prevents or combats infection. Dressings may be utilized to contain exudate. Dressings may minimize distress or disturbance to the patients, or otherwise hide or cover the wound for cosmetic reasons.

It is acceptable to combine a primary and secondary dressing, but it has become common practice to mix wound dressings and sometimes combine three or more products. This practice can be unsafe and may give rise to unnecessary extra costs. It also demonstrates a lack of knowledge and insight into why, when, and how a particular dressing product should be used. Inappropriate care can lead to delayed wound healing for patients and unnecessarily high costs for the healthcare provider.

Practitioners must identify the most suitable and cost-effective options which are maintainable by the caregivers, particularly if those caregivers are non-professionals. If bandaging is too costly or cumbersome to apply and remove, patients will delay or avoid changing bandages. If bandaging is not sufficiently resilient, it will shift with movement. Shifting bandaging may fail to protect the wound or worse cause premature debridement resulting in excessive scaring, and/or pain to the patient.

Practitioners and non-professionals alike may bind dressing too tightly or too loosely, both of which is undesirable in terms of patient treatment. Dressings which are too loose may allow foreign matter into a wound, leading to infection. Dressing which are too tight, or which cause repetitive trauma may lead to arterial insufficiency ulcers. Dressing which do not allow sufficient padding or that fail to prevent shear forces or friction may lead to decubitus ulcers (a/k/a bed sores or pressure ulcers). Once applied, especially complex multipart bandages are difficult to adjust and may even have to be completely replaced, particularly if intended for one time applications.

With current bandaging/dressing options, it is virtually impossible for a patient to apply their own compression wraps correctly. Additionally, one cannot properly handle the placement and securing of multiple bandages on multiple wound sites of close proximity. Even if a patient could properly reach all areas of an affected extremity, it is impossible to wrap gauze or elastic bandaging and hold the multiple dressings in place, and assure proper tensioning of the bandage.

What is needed is a bandaging/dressing system which allows Practitioners to specify exactly the application of treatment options, in an easy to manage and repeatable fashion by skilled and unskilled care givers. The bandaging/dressing system should not require extensive knowledge or training for correct application. The bandaging/dressing system should account for multiple treatment options simultaneously. It is especially desirable if this system is low cost and at least semi reusable.

Additionally, there is a need for a practitioner to be able to specify bandaging/dressing parameters in a wound treatment regiment which changes over time, without the patient and caregiver having to return to the practitioner for additional demonstrations and examples.

### BRIEF DESCRIPTION OF THE DRAWINGS

FIG. 1 illustrates the foot of a patient with a Wound Dressing System in accordance with an exemplary embodiment of the invention.

FIG. 2A illustrates a Primary Dressing Support in accordance with an exemplary embodiment of the invention.

FIG. 2B illustrates components of a Dressing Support in accordance with an exemplary embodiment of the invention.

FIG. 3 illustrates a Secondary Dressing Support in accordance with an exemplary embodiment of the invention.

FIG. 4 illustrates possible bandage placement of a Primary Dressing Support in accordance with an exemplary embodiment of the invention.

FIG. 5 illustrates possible bandage placement of a Secondary Dressing Support in accordance with an exemplary embodiment of the invention.

FIG. 6A illustrates a cross section of a side view of a Secondary Dressing Support to illustrate optional arch support in accordance with an exemplary embodiment of the invention.

FIG. 6B illustrates a cross section of a side view of a Dressing Support System in accordance with an exemplary embodiment of the invention.

FIG. 7 illustrates an assortment of dressings for use with a Dressing Support System in accordance with an exemplary embodiment of the invention.

### DETAILED DESCRIPTION OF THE PREFERRED EMBODIMENTS

Described herein is a dressing support system for bandaging of patient wounds. The system comprises a dressing support, which is particularly shaped to fit one or more parts of the patient's body. The dressing support system is configured to be adjustable to persons of different physical characteristics, and/or different treatment needs. That is to say, the system may be adjustable to fit a smaller person, or to provide additional compression therapy for a wound treatment regimen. Additionally, the system may be adjustable to fit a larger person, or to allow larger dressings or bandages with bulk for absorbency, or padding to be utilized without compression for regimen not requiring such.

Typically, a dressing covers a wound, and a bandage holds it in place. However, this distinction has often blurred with the advent of adhesive bandages which include wound dressing and bandage in a single element, and disposable absorbent elastic wraps which may serve as a dressing by application directly to a wound and which may be wrapped around a body part, and later discarded. Therefore, the distinction between the two is not always clear. The terms may be used interchangeably within to mean any protection, covering, or padding for aiding in healing and protecting a wound. Where distinction between the two is necessary, the context of use will dictate the purpose as that of covering a wound or securing dressings in position.

The term practitioner is utilized herein to designate a skilled provider of wound treatment. It may include, but is not limited to a physician, medical practitioner, registered nurse (RN), intern, emergency medical technitions (EMT), hospital or nursing care workers, wound care specialist, etc. The term caregiver is utilized herein to designate anyone providing care to the patient. It may include, but is not limited to practitioners, as described above, unskilled workers, family members, or the patient themselves. The Term patient is utilized herein to designate the person experiencing medical issues and thus requiring treatment of some degree. Treatment may include, but is not limited to professional care by practitioners, layman care by unskilled caregivers, or self-treatment.

The dressing support system has markings to indicate the proper placement of bandages and medications for proper application to the body when the support system enwraps the patient's body part. This allows a practitioner to provide detailed instructions which indicate the type of dressing to place in the dressing support system, to indicate the exact placement and orientation of the dressing, and any ointments or medications to be placed on the dressings. The dressing support system is then positioned on the patient's affected body part such that the dressings will properly align with wounds and provide efficient healing environments.

As an example, a practitioner may direct the patient to use absorbent pads at position X, replacing every four hours, until the wound stops weeping, then replace to a semi-permeable film dressing centered at position X and extending left and right over positions Y and Z, replacing every 12 hours until the next follow up visit. In the previous example, positions X, Y, & Z would be representative of proximately contiguous locations indicated by marking on the dressing support system which facilitate proper placement of the dressings such that they align with wounds on the patient's body when applied thereto. By following the instructions, a patient efficiently provides the correct healing environment for a wound as the wound's environment needs change, and in doing so may extend the time between visits to the practitioner.

The dressing support system is configured to wrap around a Patient's body part and secure with reusable fastening closures. The reusable closures utilized in the preferred embodiment are hook and loop fasteners, e.g. similar to those commonly known as Velcro©. One skilled in the art would appreciate that other reusable closures may be implemented such as snaps, buttons, adhesives, cling wraps, etc. The primary characteristic being an ability to remove and reposition the support system such that the dressing support system is reusable at least once. The ability of repositioning allows monitoring and adjustment of compression or bandaging without having to discard and start over or to accommodate increased or decreased localized swelling.

Dressings such as the common Band-aid® brand use adhesive coverings which adhere to the skin surrounding a wound. Another method of dressing a wound is to use separate bandaging such as adhesive tapes, coverings, or wraps to secure the dressings over the wound. Both of these are inappropriate in certain situations as it may be harmful to surrounding tissue, or multiple wounds may be proximally positioned as to not allow a clear area for the tape without harming damaged tissues. The preferred method is to position the dressing and secure said dressing without causing further damage to surrounding tissues. This is ordinarily accomplished by dressing each wound, then securing the dressings with elastic bandages. Doing so is a difficult endeavor if there are multiple dressings to position while wrapping bandages, particularly when self-applied.

In the preferred embodiment, the dressing support system is tailored for use on the foot/ankle area. Rising diabetes in America and the foot care issues which accompany it has caused an emerging need for such devices. One skilled in the art would appreciate that similar dressing systems could be utilized for the calves, thighs, elbows, hands/wrists, shoulders, etc. The system is particularly suited for extremities due to movements which render proper dressing of wounds thereon difficult, and the difficulty of reaching the feet or loss of a hand's use when self-applying to the feet or a hand, respectively.

The dressing system comprises a dressing support which is customized to enwrap the targeted body part and account for movement, similar to an orthosis, but without specifically being tailored to provide orthotic forces. In the preferred embodiment, the dressing system comprises a two piece dressing support, a primary dressing support which enwraps the ankle region and the mid-foot area (also referred to as the upper metatarsal area), and an optional secondary dressing support which is adjustably attached to the primary and enwraps the fore-foot area (also referred to as the lower metatarsal area and the phalanges).

By utilizing a hook and loop fastener to attach the secondary dressing support to the first dressing support, the attachment point can be adjusted to lengthen or shorten the distance between a heel support region and a ball support region to fit feet of different sizes. Proper attachment is accomplished by markings on the two dressing supports which proximately correspond to different “shoe sizes” or other indicators to allow accurate repetitive attachment of the separate components.

The dressing supports in the preferred embodiment are constructed from a neoprene fabric because of its stretch, durability, and resistance to chemicals, and the dirt and oils of the body. The dressing supports utilize hook and loop fasteners to allow multiple attachment points and adjustments. One skilled in the arts would appreciate that the dressing supports may be made of any flexible fibers which may be woven, pressed or knitted into sheets and/or shaped.

The dressing supports comprise markings for joining primary and secondary supports or ancillary supports. The markings allow for accurate repetitive attachment of the plurality of supports. This provides adjustments to accommodate patients of different sizes, or different treatment regimen, such as decreasing sizes to provide more compression, or increasing sizes to allow bulkier absorbent dressings or paddings.

The dressing support may be treated to have antimicrobial properties. In the preferred embodiment, the dressing support is impregnated with at least one silver ion containing compound (Ag+) and a binding compound. The dressing support in the preferred embodiment is separable from dressing materials and is washable. The dressing support may further include a removable walking sole which attaches to the outside of the dressing support and protects it from abrasions, dirt and oils from the ground. In the preferred embodiment, the walking sole is a molded rubber sole with one component of hook and loop fastener attached to the upper surface in at least one location, and mating with the complementary component of hook and loop fastener located in at least one location of the bottom of the dressing support. It is preferable that the loop portion of the hook and loop fastener be located on the dressing support and the hook portion on the upper surface of the walking sole to allow use of the dressing support without the sole.

In an embodiment of the innovation, the dressing may further include padding or cushion to facilitate use of the extremity. For instance, an arch support may be incorporated into either the primary or the secondary dressing support. In the preferred embodiment, an arch support is incorporated in the secondary dressing support to allow the primary dressing support to be worn alone, with a shoe, or other foot covering. Experimentation shows that the secondary dressing support is too bulky to fit into a patient's normal footwear and therefore should include arch support, as the patient is less likely to wear additional footwear when the secondary dressing support is utilized. Further, since the secondary dressing support is adjustable with respect to attachment to the primary dressing support, doing so allows for more flexibility for the placement of the arch and improved patient comfort.

The dressing support further comprise markings for bandage placement. The markings are individually identifiable so that they may be utilized for accurate repeatability in bandage placement. Further, individually identifiable markings allow practitioners to clearly and precisely designate placement of bandages such that the bandages will align with specific portions of the body or wound locations when the dressing is worn correctly by the patient. In one embodiment the markings may be lettered, in another embodiment, the markings may be numbered, in another embodiment the markings may be medical names for the corresponding position on the body.

Dressings utilized with the dressing support system comprise a shaped dressing. The shaped dressing may be formed to fit particular wound types or particular locations on the body. In one embodiment the dressings may be substantially rectangular; in another embodiment the dressings may be substantially elliptical; in another embodiment the dressing may comprise a pre-scored line allowing for separation of a larger dressing into several smaller dressings, or for the customization of a dressing's shape. In another embodiment the dressings may be uniquely shaped to suit a specific treatment purpose, for instance, a dressing may be cup shaped to fit over a projecting surface on the body. Examples include, but are not limited to a heel, an ankle bone, a corn, or blister. In another embodiment, the dressing may be pre-treated with particular topical medications including over-the-counter or non-prescription and prescription medications. In another embodiment the dressings may be pre-treated with particular coatings such as waxes, or hydrogels to prevent or promote sticking, and dry or wet the area depending on prescribed treatment regiments.

In one embodiment, the dressings may be hydrocolloid dressings as a barrier over excoriated skin, or for pressure ulcers, surgical wounds, abrasions, or minor burns where they facilitate rehydration and debridement of dry, sloughy, granulating, and necrotic wounds. In another embodiment, the dressings may be Hydrogel dressings in sheet form where absorbtion of exudate is necessary to facilitate autolysis or debridement of devialised tissue from the healthy wound bed. Hydrogel dressings require a secondary backing to prevent moisture from releasing into the dressing support system. In each of these dressing types, the dressing should have an adhesive back to facilitate placement of the dressing to the dressing support system. In another embodiment, a two sided adhesive may be separately employed with generic dressings, allowing them to be utilized with the dressing support system described herein.

Other dressings may be best adhered to the patient rather than the dressing support system, but would be difficult to apply. Semi-permeable film dressings utilized for their moisture or vapor and gas permeability are suitable for superficial and shallow wounds, such as those in the final states of healing which still need protection of the newly epithelialized wounds from trauma. In such a case, the dressing may have a temporary adhesive on the back side which adheres to the dressing support system until it is positioned on the patient, at which time the thin film dressing adheres to the patient with a stronger attraction than the temporary adhesive. This is useful in allowing a patient with difficulty reaching certain portions of their own body to continue to self-apply treatment.

Deodorizing dressings may be utilized for the management of fungating and malodorous wounds. An example includes, but is not limited to Metronidazole, a nitroimidazole antibiotic medication used to fight anaerobic bacteria. With such dressings, the dressing support system provides a secondary covering and containment to prevent contamination of surrounding materials such as wheel chairs, beds, etc.

In one embodiment, the dressing support has attachment points for ancillary devices such as stiffening elements, or straps for slings or traction apparatus, or restraints. In another embodiment, the dressing support may comprise attachment points for medical devices utilized in localized treatment such as an electrotherapy pain relief device.

In another embodiment, the dressing support system may be colored externally for various indications. Coloring the dressing support system red or orange, for example, makes it highly visible to prevent accidental bumping jostling, or other trauma. Coloring the dressing yellow, or including other graphic indicators may alert caregivers to the presence of possible infectious disease such as HIV, necrotizing fasciitis, or other situations requiring special precautions. Coloring the dressing flesh tones may allow the presence of an injury to go unnoticed on someone who is self-conscious. Other colors or graphics could be utilized to allow a sense of individual style, or to encourage children or other uncooperative patients to allow the wound to remain properly bandaged.

The structure of the dressing support system, the marking indicators inside and out, allow for a repeatability by even the unskilled caregiver to deliver the proper environment to the wound for proper wound care. The reusability of the dressing support system and washability allows a patient to save cost by having two systems where one can be washed while the other is utilized, and only minimal dressings need to be discarded and replaced between re-dressings. This cost savings will promote proper wound care over attempts to save money, cost, and effort due to the inefficiency of existing wound dressing protocols.

The Wound Dressing System may further include coatings in one or more locations on the outside surface of the dressing support to allow caretakers to write notes such as when the dressing was last changed. This surface should be of a rewritable fashion so that notes can be erased and replaced as further treatment continues. Such a system would be useful in a location such as a hospital or nursing care facility where multiple caretakers treat a single patient. In the preferred embodiment, the wound dressing system has a flexible plastic note card stitched to the outside of the dressing support which can be written on and erased using dry-erase markers. The notecard is positioned at a location where the patient is less likely to rub or wipe the card, or alternatively may include a clear cover which is removed to write on the notecard, then positioned to protect the notecard.

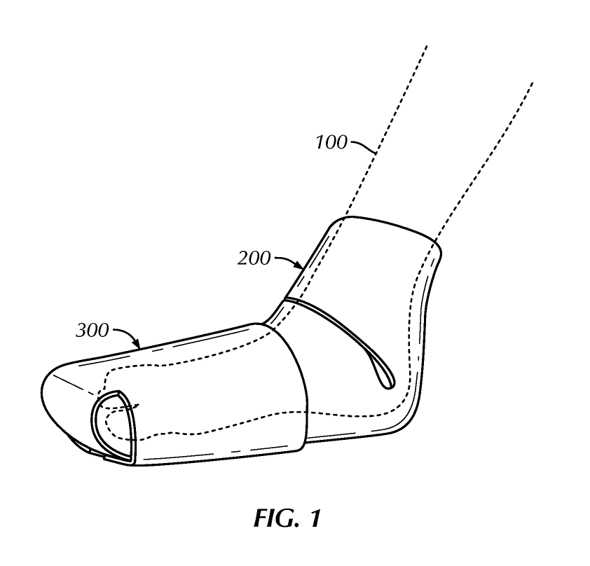

FIG. 1 illustrates the foot of a patient with a Wound Dressing System in accordance with an exemplary embodiment of the invention. A patient's foot (100) is shown wrapped in a primary dressing support (200) and a secondary dressing support (300). The primary dressing support (200) is in the proximal position, and the secondary dressing support (300) is distal position.

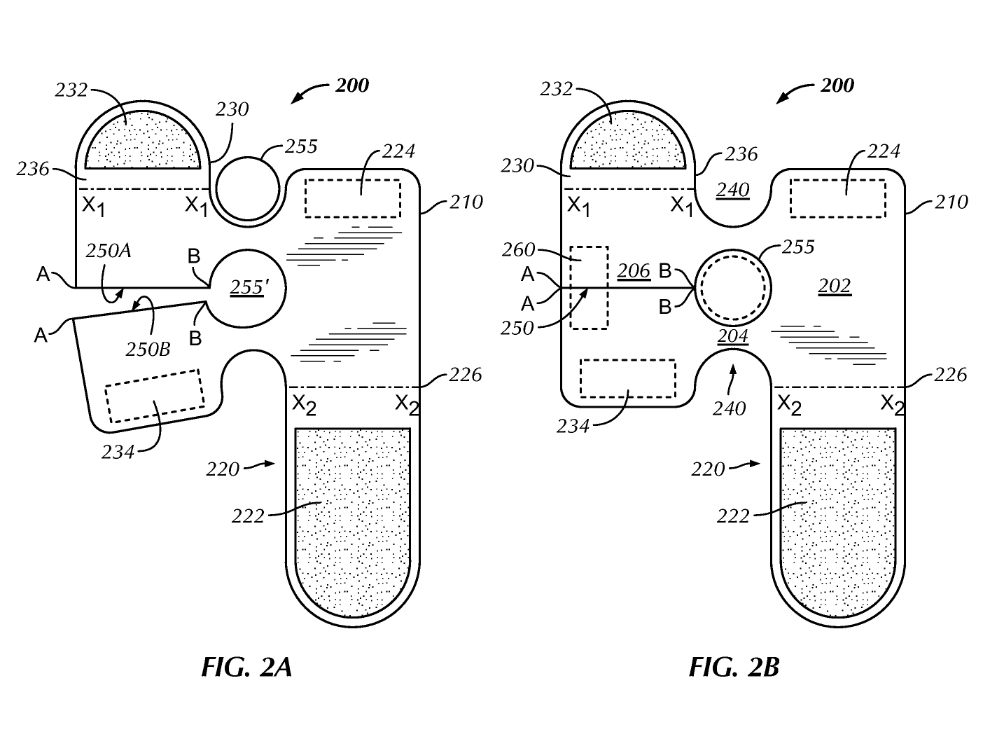

FIG. 2A illustrates a Primary Dressing Support in accordance with an exemplary embodiment of the invention. The support body (210) is elongated on one side [shown as the lower side in the drawings orientation] to form a first tab (220) that creates an ankle bandage. The actual side to which the elongation occurs would depend on whether the support was made for a left or right foot. In another embodiment, elongations may extend from either side such that they overlap in the front rather than on a side, thus producing a substantially asymmetrical object which is ambilateral in a single design which may be utilized for either the left or right.

The first tab (220) is designed to wrap around the lower tibia and fibula area of the leg allowing the tab closing material (222) to mate with the tab mating material (224). In the preferred embodiment, the tab closing material (222) and the tab mating material (224) are hook and loop type fasteners. The first tab (220), in the preferred embodiment is made of a secondary piece of fabric to avoid waste in the manufacturing process. The tab (220) is joined to the support body (210) via a seam (226) indicated as running X₂-X₂ in the drawing. Optionally the entire support body (210) may be cut from a single piece of base fabric, eliminating the need for the seam (226).

One or more shaped inserts (255) are separate from the support body (210) and larger than one or more substantially similarly shaped openings (255′) in the support body (210) such that when the edges of the shaped inserts (255) are joined to the corresponding shaped openings (255′), they force the support body (210) from a planer orientation to a desired orientation to facilitate mating with specific body components. The desired orientation may further be achieved by one or more flexing voids (240, labeled on FIG. 2B). One skilled in the arts would appreciate that the opening may be larger or smaller than the insert, and may be larger in one orientation or portion while being the same size or smaller in another orientation or portion, and may be proximally spaced in conjunction with one or more flexing voids (240) to achieve the desired deformation of the support body.

In the preferred embodiment illustrated for a foot, the shaped insert (255) is substantially circular, and has a corresponding slightly larger circular opening (255′) so that joining the edges of the shaped insert (255) to the edges of the circular opening (255′) forces the support body (210) into a cupped shape, further aided by the flexing voids (240) on either side of the shaped opening (255′) which fits the heel of the foot.

The support body (210) may further comprise one or more shaping cuts (250) shown as 250A and 250B which are mated at points AA and BB to further force the support body (210) from a planer orientation to a desired shape to fit a body part.

In the illustrated primary dressing support (200) of FIG. 2A, a second tab (230) extends to one side of the support body (210) to produce an mid-foot bandage. With the ankle bandage, created by the first tab (220) and the mid-foot bandage, created by the second tab (230); the shaped area created by the shaping insert (255) and the flexing void (240) form a hind-foot bandage which covers the heel.

The second tab (230) is designed to wrap around the upper metatarsals and the distal end of the tarsus, allowing the tab closing material (232) to mate with the tab mating material (234). In the preferred embodiment, the tab closing material (232) and the tab mating material (234) are hook and loop type fasteners. The second tab (230), in the preferred embodiment is made of a secondary piece of fabric to avoid waste in the manufacturing process. The tab (230) is joined to the support body (210) via a seam (236) indicated as running X₁-X₁ in the drawing. Optionally the entire support body (210) may be cut from a single piece of base fabric, eliminating the need for the seam (236).

FIG. 2B illustrates components of a Dressing Support in accordance with an exemplary embodiment of the invention. The primary dressing support (200) illustrated comprises three main sections: the subtalar joint support (202), the hind-foot support (204) and the mid-foot support (206). The subtalar joint support (202) enwraps the ankle area. The hind-foot support (204) is a narrowed central portion of the support body (210) which is cup shaped due to the forces exerted on the support body (210) material by the shaping cuts (250), the shaping insert (255) and the flexing voids (240) to provide bandage coverage to the heel area. The mid-foot support (206) enwraps the upper metatarsal area of the foot, also known as the mid-foot.

In this figure, the shaping cuts (250) are shown joined to align points AA and BB, and extension mating material (260) has been stitched to the back of the support body (210) spanning over the joined shaping cut (250).

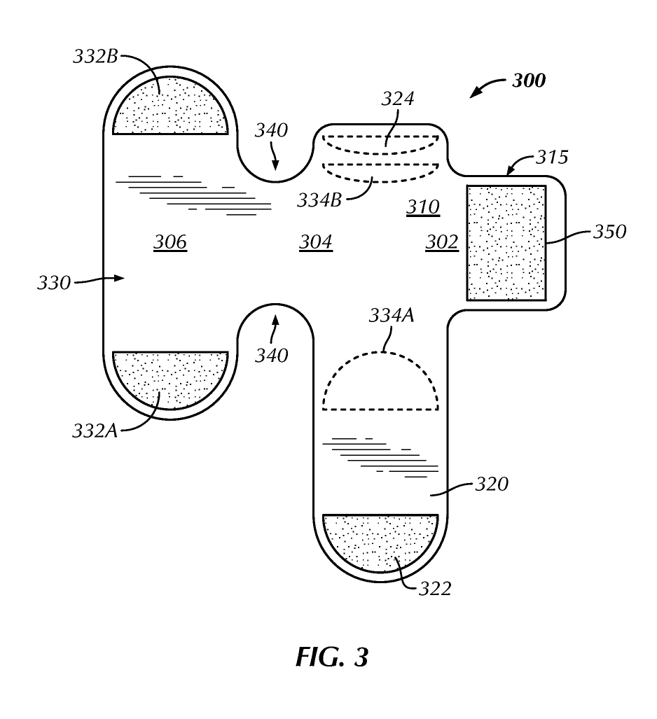

FIG. 3 illustrates a Secondary Dressing Support in accordance with an exemplary embodiment of the invention. The secondary dressing support (300) comprises three primary areas: the metatarsal support area (302), the phalanges lower support area (304) and the phalanges upper support area (306). The metatarsal support area (302) bandages the lower metatarsal area. The phalanges lower support area (304) bandages the bottoms of the phalanges, and the phalanges upper support area (306) provides bandage coverage to the upper phalanges.

The support body (310) of the secondary dressing support (300) joins to the primary dressing support (200, not shown) by means of the adjustment tab (315) which has a mating material (350) that mates to the extension mating material (260). By overlapping more or less of the adjustment tab's (315) mating material (350) with the extension mating material (260), the overall length of the primary dressing support (200) and the secondary dressing support (300) combination can be altered to accommodate patients with different sized feet, or different compression requirements or bandaging needs.

The support body (310) is elongated on one side [shown as the lower side in the drawings orientation] to form a first tab (320) that creates a bandage which enwraps the lower metatarsal area of the fore-foot. The actual side to which the elongation occurs would depend on whether the support was made for a left or right foot. In another embodiment, elongations may extend from either side as discussed previously to produce a substantially asymmetrical object which is ambilateral such that a single design may be utilized for either the left or right.

The first tab (320) is designed to wrap around the lower metatarsal area of the fore-foot allowing the tab closing material (322) to mate with the tab mating material (324). The first tab may be made of a secondary piece of fabric to avoid waste in the manufacturing process, and joined by means of a seam as was previously described above. Optionally the entire support body (310) may be cut from a single piece of base fabric, eliminating the need for a seam, as shown in FIG. 3.

One or more flexing voids (340) allow for folding of the support body (310) back over itself and into a desired orientation (shown in other FIG. 1) without gathering or bunching of the support body fabric. In the preferred embodiment illustrated for a foot, the flexing voids (340) are spaced to create a second tab (330) which extends from the support body (310) distal the adjustment tab (315). This allows for the second tab (330) of the support body (310) to be folded over between the phalanges lower support area (304) and the phalanges upper support area (306) to provide protection to the ends of the phalanges, and a possible additional dressing position. This also allows the tab closing material (332A, 332B) to be folded under the foot to align with the tab mating material (334A, 334B), thus closing over the end of the phalanges.

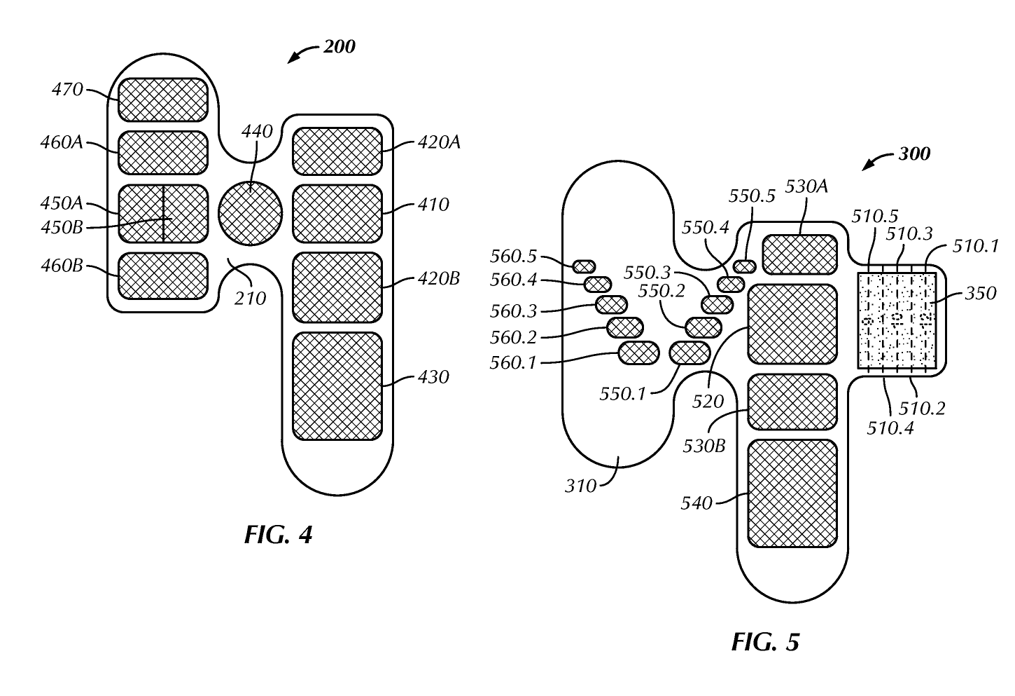

FIG. 4 illustrates possible bandage placement of a Primary Dressing Support in accordance with an exemplary embodiment of the invention. Markings of various bandage placements are uniquely suited to the particular application of the embodiment. That is, markings for a foot bandage would not necessarily be the same in name or location as those for an elbow bandage or a knee bandage. Bandage locations may be indicated by labels, or placement indicators (410-470) on the support body's (210) material surface. The primary dressing support (200) illustrated herein comprises the following bandage placement locations which can be marked with letters, numbers, or other indicia. In the preferred embodiment, the locations are printed with medical terms descriptive of the area covered, and serve to train practitioners and non-practitioners, or to allow bandaging in compliance with a doctor's regular written orders without need of translation to layman's terms.

The Posterior Tarsus (410) position is for wounds on the back of the ankle. The Lateral Tarsus (420A) position is for wounds on the outside of the ankle. The Medial Tarsus (420B) position is for wounds on the inside of the ankle. The Dorsal Tarsus (430) position is for wounds on the front of the ankle area.

The Posterior Calcaneus (440) position is for wounds on the heel area. The Distal Calcaneus (450B) position is for wounds on the front bottom of the projection of the heel. The Plantar Metatarsal (450A) position is for wounds on the arch. The Lateral Proximal Metatarsal (460A) position is for wounds on the outside of the arch. The Medial Proximal Metatarsal (460B) position is for wounds on the inside of the arch. The Dorsal Proximal Metatarsal (470) position is for wounds on the top of the foot, near the ankle

FIG. 5 illustrates possible bandage placement of a Secondary Dressing Support in accordance with an exemplary embodiment of the invention. The size adjusting tab (315, not marked) uses markings on the tab (315) and/or the adjustment tab mating material (350) to facilitate the accurate and repeatable joining of the secondary dressing support (300) to the primary dressing support (200). Indicators (510.1-510.5) show where to align the edge of the primary dressing support (200). In the preferred embodiment, the indicators show shoe sizes. Position 510.1 is indicated, in the example shown, as being proper placement for a patient with a size 12 foot. Position 510.5 is indicated, in the example shown, as being proper placement for a patient with a size 9 foot. Other embodiments may be sized differently, and would be marked appropriately to the proper sizing for the specific embodiment.

As previously discussed, markings of various bandage placement is uniquely suited to the particular application of the embodiment. Bandage locations may be indicated by labels, or placement indicators (520-560) on the support body's (310) material surface. The primary dressing support (300) illustrated herein comprises the following bandage placement locations.

The Metatarsal Region (520) position is for wounds in the area of the ball of the foot. The Lateral Distal Metatarsal (530A) position is for wounds on the outside of the ball of the foot. The Medial Distal Metatarsal (530B) position is for wounds on the inside of the ball of the foot. The Dorsal Distal Metatarsal (540) position is for wounds on the top of the foot near the toes.

The Plantar Great Phalange (550.1) position is for wounds on the bottom of the great toe. The Plantar Phalange (550.2-550.5) positions are for wounds on the bottom of the lesser toes. The Dorsal Great Phalange (560.1) position is for wounds on the top of the great toe. The Dorsal Phalange (560.2-560.5) positions are for wounds on the top of the lesser toes.

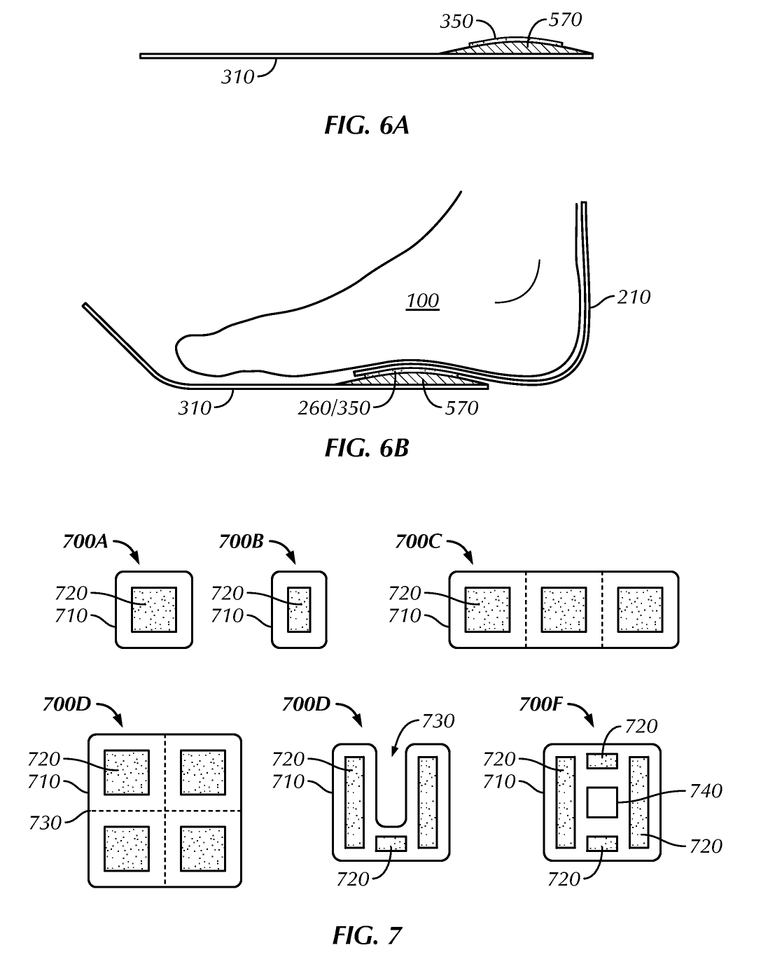

FIG. 6A illustrates a cross section of a side view of a Secondary Dressing Support to illustrate optional arch support in accordance with an exemplary embodiment of the invention. The secondary support body (310) has a built up area which serves as an arch support (570) to aid walking without shoes while utilizing the dressing support system. The adjustment tab mating material (350) is shown to indicate that the arch support would be located in the area of the size adjusting tab (315, not indicated).

FIG. 6B illustrates a cross section of a side view of a Dressing Support System in accordance with an exemplary embodiment of the invention. The arch support (570) is positioned correctly at the arch of the patient's foot (100) when the adjustment tab mating material (350) of the secondary dressing support (310) is properly aligned with the extension mating material (260) of the primary dressing support (210).

FIG. 7 illustrates an assortment of dressings for use with a Dressing Support System in accordance with an exemplary embodiment of the invention. A standard dressing may be rectangular (700A) or elliptical (700B). Dressing primarily comprise the dressing material (710) which is wound specific, and an adhesive pad (720) which is used to adhere the dressing to the dressing support system. Sizable dressings (700C and 700D) further comprise a sizing or shaping cut (730) which is a pre-scored or creased marking which can help in cutting, tearing, or otherwise parting a portion of the dressing for a specific application.

Irregular dressings (700D) are utilized for specific areas of the body. Like standard or sizable dressing, they again have dressing material (710) and one or more adhesive points (720), but may also include shaping voids (730) which allow them to better fit an area. Specialized dressings (700F) may additionally contain a void (740) which may be an opening in the dressing, or a thinner area, or may contain medications.

The diagrams in accordance with exemplary embodiments of the present invention are provided as examples and should not be construed to limit other embodiments within the scope of the invention. For instance, heights, widths, and thicknesses may not be to scale and should not be construed to limit the invention to the particular proportions illustrated. Additionally, some elements illustrated in the singularity may actually be implemented in a plurality. Further, some elements illustrated in the plurality could actually vary in count. Further, some elements illustrated in one form could actually vary in detail. Further yet, specific numerical data values (such as specific quantities, numbers, categories, etc.) or other specific information should be interpreted as illustrative for discussing exemplary embodiments. Such specific information is not provided to limit the invention.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A wound care system comprising;
a dressing support and
one or more dressings, wherein the dressings are positioned on the dressing support system to align with treatment locations on a patient when the dressing support is applied to the patient.

2. A wound care system, as described in claim 1 wherein the dressing support further comprises a washable reusable fabric.

3. A wound care system, as described in claim 2 wherein the washable reusable fabric is a fabric backed neoprene.

4. A wound care system, as described in claim 2 wherein the dressing support is impregnated with at least one silver ion containing compound.

5. A wound care system, as described in claim 1 wherein the one or more dressings are temporarily attached to the dressing support prior to the dressing support being applied to the patient.

6. A wound care system, as described in claim 2 wherein the dressing support is uniquely colored to correspond with targeted utilization environments.

7. A wound care system, as described in claim 1 wherein the dressing support further comprises closable tabs which are removably attached to opposing closable tabs which are adjustable to custom fit a patient.

8. A wound care system, as described in claim 1 wherein the dressing support further comprises a primary dressing support and a secondary dressing support.

9. A wound care system, as described in claim 8 wherein the primary dressing support and the secondary dressing support are removably connected and adjustable to accommodate a plurality of patient sizes.

10. A wound care system, as described in claim 1 wherein the dressings comprise:
a substantially flat dressing material having a front side and a back side,
wherein the front side is designated to orient toward a patient injury site, and
the back side is designated to orient away from a patient injury site, and toward the dressing support;
a binding material applied to the back side of the dressing;
an optional backing material temporarily covering the binding material.

11. A wound care system, as described in claim 10 wherein the dressing is optionally coated on at least the front side with one or more topical medications.

12. A wound care system, as described in claim 10 wherein the front side is optionally coated with one or more wound treatment conditioners.

13. A wound care system, as described in claim 10 wherein the dressing is partially cut or pre-stressed to facilitating parting into one or more smaller dressings.

14. A wound care system, as described in claim 10 wherein the dressing further comprises:
one or more voids or cuts to facilitate shaping the substantially flat dressing to a non-planer orientation.

15. A wound care system, as described in claim 1 wherein the dressing support further comprises:
ancillary attachment points for wound care accessory items.

16. A wound care system, as described in claim 15 wherein the ancillary attachment points are connector points and the wound care accessory items are straps to form a sling.

17. A wound care system, as described in claim 1 wherein the dressing support comprises rewritable external surfaces to facilitate caretaker notes near a wound site.

18. A method of utilizing a wound care system comprising:
specifying a dressing support to correspond with a patient's injuries and physical characteristics;
specifying dressing types for one or more patient injuries;
specifying medications and/or conditioners for application on the one or more dressing types; and
specifying application locations for the dressings which correspond to marked locations on the dressing support.

19. A method of utilizing a wound care system, as specified in claim 18 further comprising:
utilizing a specified dressing support;
optionally applying medications and/or conditioners to specified types of dressings;
applying dressings to the dressing support in specified locations;
applying the dressing support to the patient.

20. A method of utilizing a wound care system, as specified in claim 18 further comprising:
utilizing dressing material;
applying a temporary binding substance to one side of the dressing;
optionally applying medication or conditioners to the dressing opposite the binding substance;
applying a temporary and removable backing to protect the binding substance until use;
optionally packaging the dressing material for later utilization.
